(19) United States
(12) Patent Application Publication
(10) Pub. No.: US 2010/0275274 A1
(43) Pub. Date: Oct. 28, 2010

(54) MOUSE MODEL FOR DEPRESSION, SCHIZOPHRENIA AND ALZHEIMER'S DISEASE

(75) Inventors: YI-MING CHEN, TAIPEI CITY (TW); CHING-PING YANG, TAIPEI CITY (TW)

Correspondence Address: Muncy, Geissler, Olds & Lowe, PLLC, 4000 Legato Road, Suite 310, FAIRFAX, VA, 22033, US

(73) Assignee: NATIONAL YANG-MING UNIVERSITY, TAIPEI CITY (TW)

(21) Appl. No.: 12/431,641

(22) Filed: Apr. 28, 2009

(51) Int. Cl. G01N 33/00; A01K 67/00; C12N 15/00

(52) U.S. Cl. 800/3; 800/9; 800/12; 800/21

(57) ABSTRACT

The present invention relates to Glycine N-methyltransferase (GNMT) animal model and use thereof.

### FIELD OF THE INVENTION

The present invention relates to Glycine N-methyltransferase (GNMT) knockout animal model and use thereof.

### BACKGROUND OF THE INVENTION

Glycine N-methyltransferase (GNMT), also known as a 4S polycyclic aromatic hydrocarbon (PAH) binding protein, has multiple functions. In addition to acting as a major folate binding protein (Yeo E J, et al. Proc Natl Acad Sci USA 1994; 91:210-214), it also regulates the ratio of S-adenosylmethionine (SAM) to S-adenosylhomocysteine (SAH) by catalyzing sarcosine synthesized from glycine (Kerr S J. J Biol Chem 1972; 247:4248-4252). It was previously reported that the GNMT gene is down-regulated in HCC (Liu H H, et al. J Biomed Sci 2003; 10:87-97). Results from a genetic epidemiological study indicate that GNMT is a tumor susceptibility gene for liver cancer (Tseng T L, et al. Cancer Res 2003; 63:647-654). In addition, it was reported that GNMT binds benzo(a)pyrene and prevents DNA-adduct formation (Chen S Y, et al. Cancer Res 2004; 64:3617-3623).

In mice, GNMT expression is regulated by growth hormone, with the hepatocytes of female mice having up to eight times the expression level normally found in male mice. There have been three reports of pediatric patients (two boys, one girl) with congenital GNMT deficiencies resulting from a missense mutation in the GNMT gene (Augoustides-Savvopoulou P, et al. J Inherit Metab Dis 2003; 26:745-759). All three children had hypermethioninaemia, clinical symptoms mimicking chronic hepatitis (Augoustides-Savvopoulou P, et al. J Inherit Metab Dis 2003; 26:745-759). The girl had stunted growth and suffered from mental deficiency (IQ 87).

The prior art disclosed a GNMT knock-out mouse which showed abnormal liver function and suffered from glycogen storage disease (U.S. application Ser. No. 11/832,304).

### SUMMARY OF THE INVENTION

The present invention provides an animal model for studying depression, schizophrenia or Alzheimer's disease, wherein the animal model is a mammal whose genome is disrupted by recombination at Glycine N-methyltransferase (GNMT) gene locus.

The present invention also provides a method of generating an animal exhibiting a pathological condition of depression, schizophrenia or Alzheimer's disease, comprising disruption of GNMT gene in the animal by recombination at GNMT gene locus.

The present invention further provides a method for screening a drug candidate for treating depression, schizophrenia or Alzheimer's disease in a subject, comprising: (a) administering a potential drug candidate to the animal model of claim 1, (b) measuring the response of said animal to said drug candidate, (c) comparing the response of said animal with that of an animal having a wild type GNMT gene, and (d) selecting the drug candidate based on the difference in response observed between said animal and said animal having a wild type GNMT gene.

### BRIEF DESCRIPTION OF THE DRAWINGS

FIG. 1 shows the strategy of constructing the targeting vector.

FIG. 2 shows targeted modification of the GNMT gene locus. (A) Targeting vector was designed to replace GNMT exons 1-4 and a part of exon 5 with a neomycin resistance gene. Neomycin positive selection marker is flanked by two homologous regions and followed by a TK negative selection marker at the 3′ end of the targeting vector. (B) Southern blot analysis of embryonic stem cell clones. BamHI (B)-BamHI DNA fragment size decreased from 7.9 kb (wild-type allele) to 5.3 kb (recombinant allele). (C) Genotyping of GNMT knockout mice by PCR. The normal GNMT allele yielded a 772 bp fragment and the disrupted allele a 409 bp fragment. +/+, wild-type; +/−, GNMT heterozygous and −/−, GNMT−/− mice (D) Expression of GNMT protein confirmed by western blot analysis. Each lane contains 10 μg hepatic lysate. GNMT molecular mass: 32 kDa. GAPDH: internal control.

FIG. 3 shows GNMT−/− male mice displayed significant deficits in prepulse inhibition of the acoustic startle reflex. Data are presented as mean±S.E.M.; **p<0.01.

FIG. 4 shows using TST and FST, GNMT−/− displayed significant increased immobility in the TST (A) and FST (B). Data are presented as mean±S.E.M.; *p<0.05.

FIG. 5 shows that no significant difference of locomotor activity was found between both sexes of WT and GNMT−/− mice.

FIG. 6 shows GNMT−/− mice having motor deficits of shorter latency of falling from the Rotating rod task. Data are presented as mean±S.E.M.; *p<0.05.

FIG. 7 shows comparing the following Alzheimer's Disease associated mRNA expression in the cerebral cortex of one-month old mice by Q-PCR, as follows, APP, BACE 1, BACE 2, APH-1α, GSK-3β, MAPT, SNCα and IDE. Data are presented as mean±S.E.M.; *p<0.05.

FIG. 8 shows that together with nestin (A), GNMT (B) expression was found on neural progenitor cell in immunofluoresencent images.

FIG. 9 shows detecting of GNMT expression in WT mouse brain (a) GNMT mRNA in different parts of mouse brain using RT-PCR. 1) olfactory bulb, 2) cortex, 3) striatum, 4) midbrain, 5) cerebellum, 6) spinal cord, 7) hippocampus, 8) thalamus and hypothalamus, 9) pons and medulla, 10) brain stem. (b) Immunostaining of GNMT in WT mice brain.

### DETAILED DESCRIPTION OF THE INVENTION

The GNMT gene expresses in the neural progenitor cell and partial region of the brain, such as cortex, straitum and substantia nigra. Besides, the Depression-like and Schizo-like behaviors were observed in the Gnmt knock-out mouse model. Furthermore, the GNMT gene expresses in the mouse neuron cells which develops in vitro. Those results indicate the GNMT gene plays essential role in the function of brain and development.

According to the experimental results, it shows that metabolite of dopamine, dihydroxyphenylacetic acid, of the GNMT knock-out mouse is apparently reduced. Besides, it is observed that the GNMT knock-out mice display significant deficits in prepulse inhibition of the acoustic startle test compared with wild-type mouse. It is also observed that GNMT knock-out mouse is easier to give up to the TST (tail suspension test) and FST (force swim test). From the RotaRod motor test, the result shows the exercise ability of GNMT knock-out mouse is inferior to that of wild-type mouse. The GNMT knock-out mouse presents Depression-like and Schizo-like behavior. Furthermore, the results of microarray show GNMT deficiency results in the increase expression of Alzheimer's disease related genes. Hence, the present animal model could be applied to research the Depression, Schizophrenia and Alzheimer's disease.

The animal model of this invention, wherein the animal includes but is not limited to mammal, primate, and rodent. In a preferable embodiment, the animal is mouse.

The present invention also provides a method of generating an animal exhibiting a pathological condition of depression, schizophrenia or Alzheimer's disease, comprising disruption of GNMT gene in the animal by recombination at GNMT gene locus. The pathological condition is characterized by deficits in prepulse inhibition of acoustic startle reflex, decreased immobility of tail suspension test and forced swim test, or elevating expression of Alzheimer's disease-associated genes. The elevating expression of Alzheimer's disease-associated genes includes but is not limited to BACE 1, BACE 2, APH-1, GSK-3, MAPT, and IDE.

The present invention further provides a method for screening a drug candidate for preventing or treating depression, schizophrenia or Alzheimer's disease in a subject, comprising: (a) administering a potential drug candidate to the animal model of the present invention, (b) measuring the response of said animal to said drug candidate, (c) comparing the response of said animal with that of an animal having a wild type GNMT gene, and (d) selecting the drug candidate based on the difference in response observed between said animal and said animal having a wild type GNMT gene.

As used herein, “drug candidate” means a composition of matter that is being investigated for a pharmacological or other activity or that is known to have a pharmacological or other activity, but is being tested to see if it has any type of activity in a particular subject, such as a patient. The drug candidate includes but is not limited to nucleic acid, peptide, and chemical compound. Efficacy of a drug candidate is one example of a pharmacological activity. Moreover, clinical outcome can be characterized as an activity of a drug candidate.

The present invention also provide a method for screening a drug candidate for treating depressing, schizophrenia or Alzheimer's disease in a subject, comprising: (a) providing a mammalian cell comprising a disruption in an endogenous GNMT gene, wherein the disruption results in a reduced level of an GNMT biological activity in the mammalian cell as compared to that of a wild type cell under identical conditions, (b) administering the potential drug candidate to the cell of step (a), and (c) comparing the response of said cell with that of a cell having a wild type GNMT gene, and (d) selecting the drug candidate based on the difference in response observed between said cell and said cell having a wild type GNMT gene. The method of the present invention, wherein the mammalian cell is present within a knockout non-human mammal, and the preferable cell is neural progenitor cell of brain.

### EXAMPLES

### Example 1

### Preparing the GNMT Knock-Out Mouse

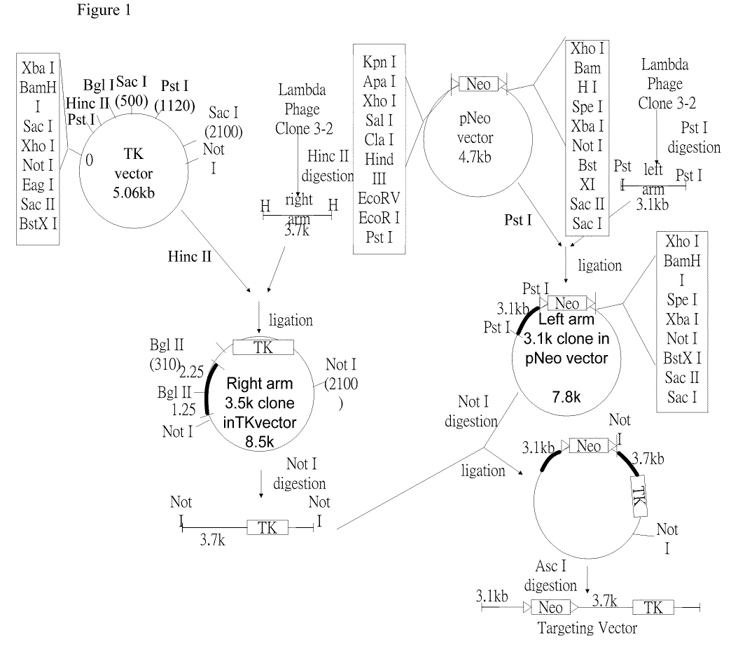

To construct a targeting vector, DNA fragments digested from lambda phage clones 3-2 and 5-3 were inserted into a plasmid-pBluescript II KS. Left arm was digested from the phage clone 5-3 by using Pst I and inserted into the pNeo vector. Right arm was digested from the phage clone 3-2 by using Hinc II and inserted into the TK vector. The fragment containing right arm and TK gene was digested by using Not I and inserted into the pNeo vector containing left arm to generate the targeting vector (FIG. 1).

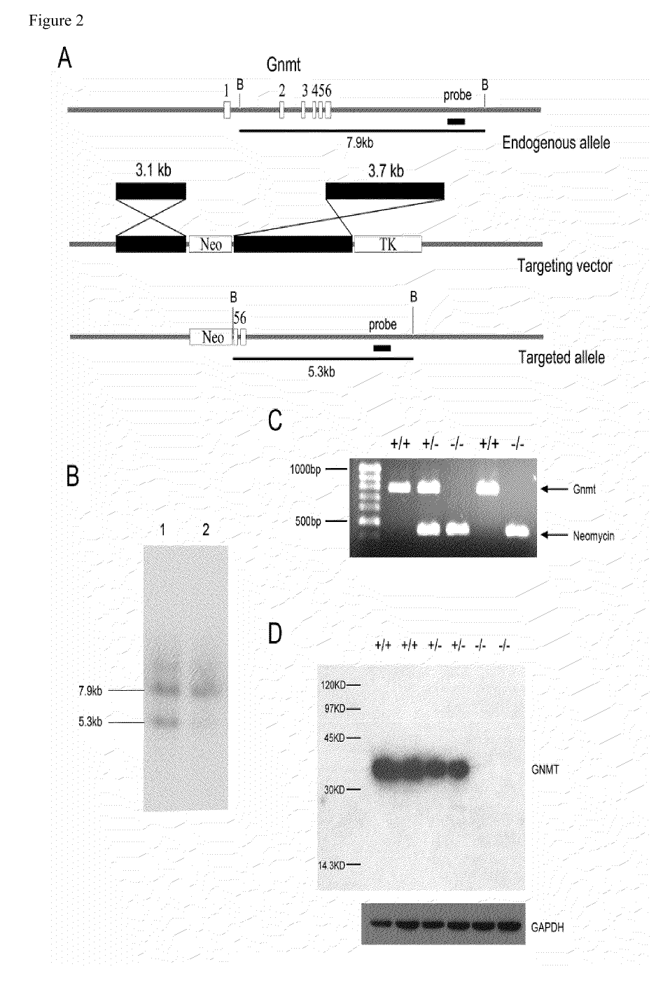

The neomycin gene (to replace exons 1-4 and part of exon 5 of the mouse Gnmt gene) was framed with two DNA fragments (3.1 kb and 3.7 kb) in the targeting vector. The thymidine kinase gene was used as a negative selection marker (FIG. 2A). The 40 μg targeting vector was linearized using AscI and introduced into embryonic stem cells (129/Sv-derived) by electroporation. After screening 278 clones using southern blot analysis (FIG. 2B), a recombinant clone was isolated and used for microinjection into blastocytes. Four male chimeric mice were obtained and used to breed female C57BL/6 mice. Agouti F₁ offspring were subjected to PCR to detect the germline transmission of the disrupted allele. Heterozygous F₁ male mice were backcross with female wild-type C57B/6 mice to generate C57BL/6 genome background mice.

PCR was developed to differentiate wild-type (+/+), GNMT heterozygous (+/−), and GNMT−/− mice. The primers used for PCR were shown as the following: GNMT-F (5′-GCGGCGGCCGCATGCTGGTGGAAGAGGGC) and GNMT-R (5′-TTGCAGTCTGGCAAGTGAGC) for GNMT; neomycin-F (5′-GTTCCTTGCGCAGCTGTGCT) and neomycin-R (5′-CGGCCACAGTCGATGAATCC) for neomycin. The normal GNMT allele yielded a 772 bp fragment by GNMT primers and the disrupted allele yielded a 409 bp fragment by neomycin primers (FIG. 2C). The expression of GNMT protein in liver was analyzed using western blot; the results show that compared to the wild-type, GNMT expression decreased approximately 50% in the livers of GNMT+/− mice and GNMT was undetectable in the livers of GNMT−/− mice (FIG. 2D).

### Example 2

### Prepulse Inhibition (PPI) of Startle Reflex

The apparatus consisted of two startle chambers (Med Associates, Georgia, Vt.). One mouse selected from the WT control group and the other selected from GNMT deficient group were tested simultaneously. Each mouse was put into the PPI chamber for a 5-min acclimatization period with a 60 dB background noise. Following this period, 10 startle pulses (120 dB, 40 ms duration) were presented with an average inter-trial interval of 15 s. Then, no stimulus (background noise, 68 dB), prepulses alone (72, 76 and 84 dB, 20 ms duration), startle pulses alone, and prepulses followed 80 ms later by startle pulses were presented six times randomly distributed over the next 20 min.

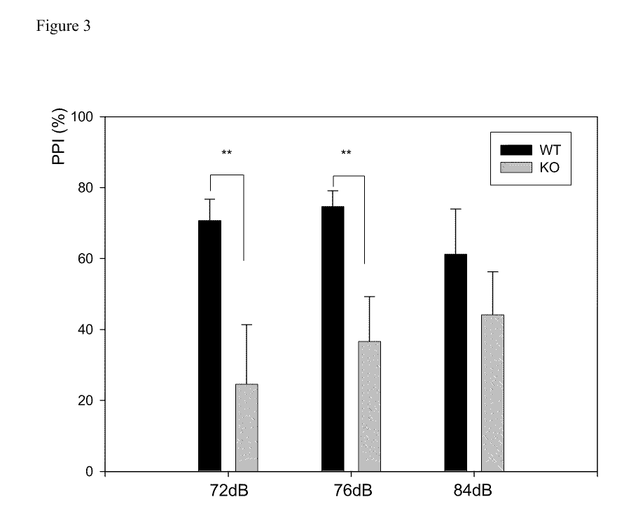

PPI was defined as the percentage reduction of startle magnitude in the presence of the prepulse compared to the magnitude in the absence of the prepulse. % PPI=[1−(prepulse trials/startle-only trials)]×100. FIG. 3 showed GNMT−/− mice displayed significant deficits in prepulse inhibition of the acoustic startle reflex.

### Example 3

### Tail Suspension Test

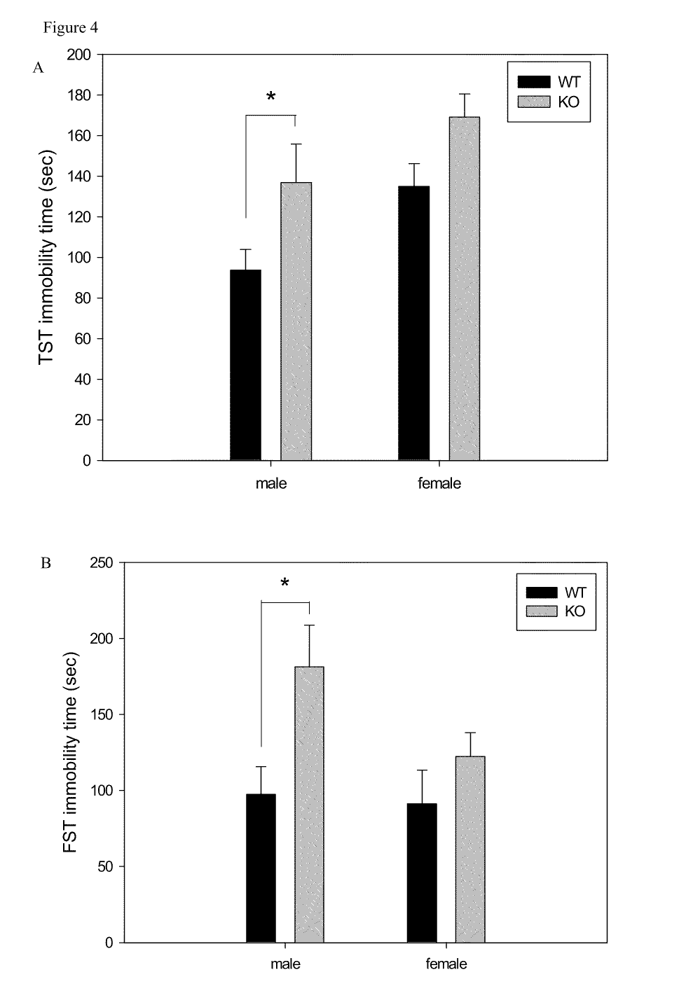

8-12 weeks old male and female mice were suspended by the tail. After ‘agitation’ or ‘escape-like’ behavior, mice adopted an immobile posture, suggested to mirror a state of depression. The immobility time during a 5 min test recorded. The result of FIG. 4A showed that GNMT−/− mice displayed significant decreased immobility.

### Example 4

### Forced Swim Test

8-12 weeks old male and female mice were placed (n=11 per WT and n=13 per KO) individually in rectangular cage (height 30 cm, diameter 15 cm) filled with 12-cm-deep water (temperature 22±1° C.) for 6 min. The processes of the total period of immobility during the last 5 min were recorded. The immobility of GNMT^{-/-} mice was decrease in the FIG. 4B. Immobility was defined as the absence of initiated movements and includes passive swaying.

### Example 5

### Analysis of Motor Activity

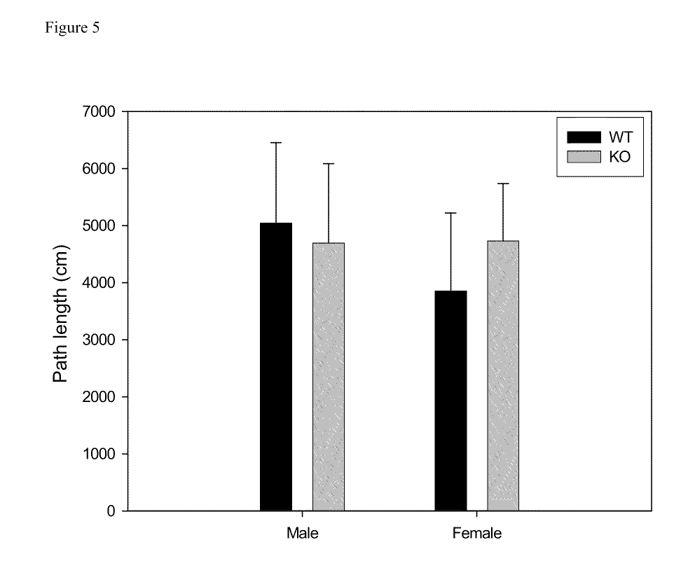

GNMT−/− animal and their wild-type were individually tested for motor activity at 8-12 weeks of age under 90 cm×90 cm×30 cm open field. Each mouse was tested for 10 min between 1700 and 1900 h. The results shown in FIG. 5 were generated online by the TrackMot software package. There was no significant difference found between both sexes of WT and GNMT^{-/-} mice.

### Example 6

### Rotarod Testing

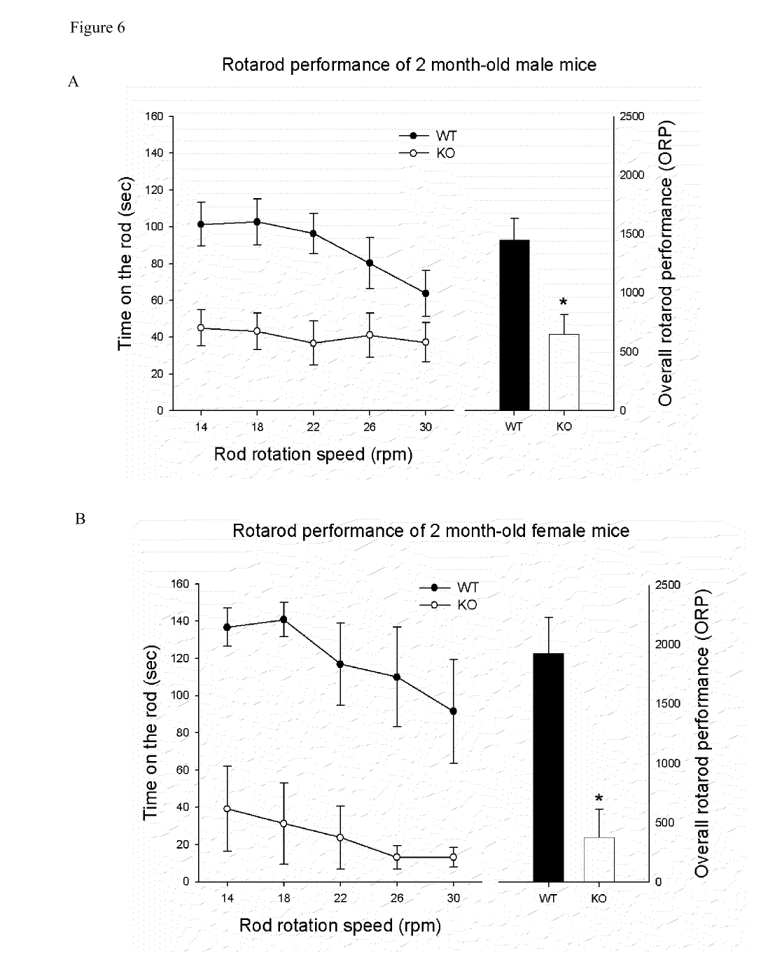

On the day of testing, all animals were transferred to the test room at least half an hour earlier. Then the mice were tested on a rotarod apparatus which consisted of a rotating rod (diameter, 3 cm; hard non-slipping plastic). All mice were habituated to the apparatus for at least four consecutive trials in which the rod was kept at constant speed (one trial at 0 rpm and three trials at 5 rpm) with 5 minutes interval. Once the trained animals were able to stay on the rod rotating at 5 rpm for 60 seconds in three consecutive trials, they proceeded to the test. Three trials at each of five fixed rotating speeds (14, 18, 22, 26, and 30 rpm) were sequentially conducted for a maximum of 150 seconds each speed or until the animals fell off. The length of time that each animal was able to stay on the rod at each rotation speed was recorded (latency to fall). Regardless of completion or fall, each animal was allowed to rest for at least 5 minutes between individual testing speeds and 30 minutes between each complete trial. The mean of overall rod performance (ORP) for the three trials of each mouse was calculated by the trapezoidal method as the area under the curve in the plot of latent time on the rod versus rotation speed. (FIG. 6)

### Example 7

### RNA Isolation and RT-PCR

Total RNA was extracted from tissues using TRIzol (Invitrogen, Carlsbad, Calif.). Complementary DNA was produced from olfactory bulb, cortex, striatum, midbrain, cerebellum, spinal cord, hippocampus, hypothalamus, medulla and brain stem RNA (5 μg) using a SuperScript II Reverse Transcriptase Kit (Invitrogen). PCR conditions were pre-denaturated at 94° C. for 5 minutes followed by 30 cycles of amplification at 94° C. for 30 seconds, 60° C. for 30 seconds, and 72° C. for 1 min, followed by a 10-minute extension step at 72° C. The primer sequences are followings: m-GNMT-F (5′-GCGGCGGCCGCATGCTGGTGGAAGAGGGC) and m-GNMT-R (5′-TTG CAGTCTGGCAAGTGAGC) for GNMT; β-actin-F (5′-GGGCGCCCCAGGCACCA) and β-actin-R (5′-CTCCTTAATGTCACGCCGATTTC) for β-actin.

### Example 8

### Real-Time PCR

Ten genes belonging to the Alzheimer Disease pathway were selected for real-time PCR analysis. Real-time PCR primers were designed using PRIMER EXPRESS software (Version 2.0, Applied Biosystems) and verified the specificity of sequences using BLAST. Reactions were performed in 101 quantities of diluted cDNA sample, primers (100 nM), and a SYBR Green PCR Master Mix containing nucleotides. Reactions were assayed using an Applied Biosystems Prism 7000 sequence detection system.

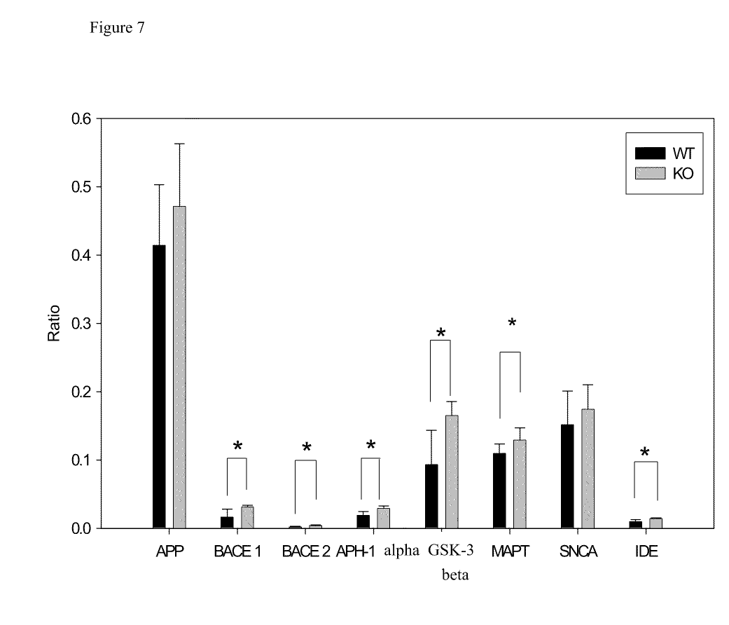

After cycling, a melting curve was produced via the slow denaturation of PCR end products to validate amplification specificity. Predicted cycle threshold (CT) values were exported into EXCEL worksheets for analysis. Comparative CT methods were used to determine relative gene expression folds to GAPDH. The primers used for real-time PCR were shown as the followings: APP-F (5′-GCCAGCCAATACCGAAAATG) and APP-R (5′-GATGTTTGTCAGCCCAGAACCT) for APP; BACE1-F (5′-ACGACTCTTTGGAGCCCTTCT) and BACE1-R (5′-AGAGCTGCAGGGAAAAGATGTT) for BACE1 for BACE; BACE2-F (5′-CACGGAAGACATAGCCAGCAA) and BACE2-R (5′-TCAGGGCATAGGACACAATCC) for BACE2; IDE-F (5′-CGTCCAATCTGATGGCGATT) and IDE-R (5′-AGAACAGCTTCACCACCAGGTTA) for IDE; SNCA-F (5′-AAACACCTAAGTGACTACCACTTATTTCTAAA) and SNCA-R (5′-TCTTGGAGCAAATCACAACTTCTT) for SNCA; MAPT-F (5′-AGCAATGAGAGATTTGAGACTTGGT) and MAPT-R (5′-CCTTCGCTGTCGCTGTTTC) for MAPT; APH1α-F (5′-ATGCACGGCTCCAGTATGG) and APH1α-R (5′-GCAAAACGGAACACTTCCTGTAG) for APH1α; GSK3β-F (5′-CGGGACCCAAATGTCAAACT) and GSK3-R (5′-TCCGAGCATGTGGAGGGATA) for GSK3β; GAPDH-F (5′-TGGTATCGTGGAAGGACTCA) and GAPDH-R (5′-AGTGGGTGTCGCTGTTGAAG) for GAPDH. (FIG. 7)

### Example 9

### Immunofluorescent Staining

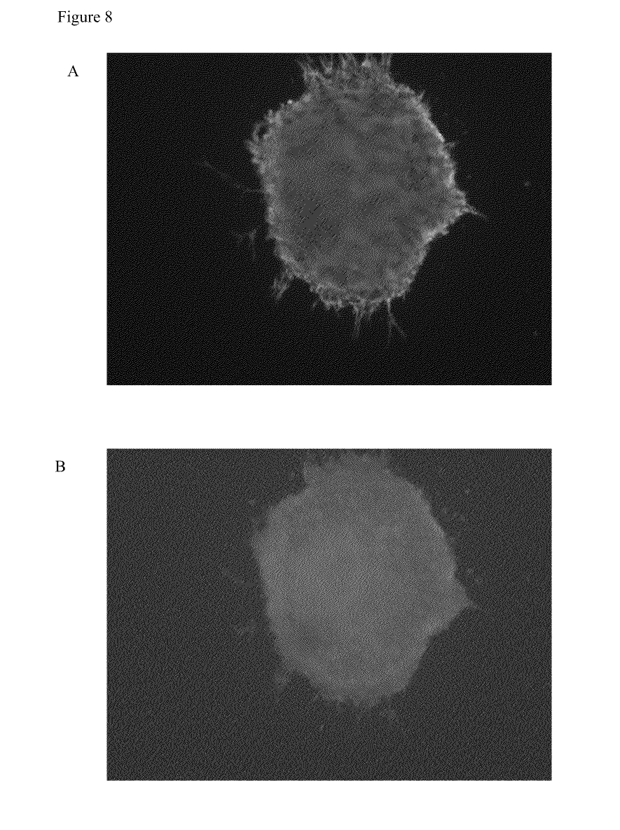

Neural progenitor cell culture followed the protocol by Zhou. et al. After 7 or 10 days in the culture of subplating, the subcultures were washed with cold 0.1 M PBS three times and then fixed with 4% paraformaldehyde for 4 h and permeated with 0.1% Triton X-100 for 30 min. The phenotypic expression of the neurospheres was examined using immunocytochemical staining with antibodies against (a) Nestin (1:500) (BD Biosciences) for neuroepithelial stem cells or (b) GNMT (1:250). (FIG. 8)

### Example 10

### Immunohistochemistry

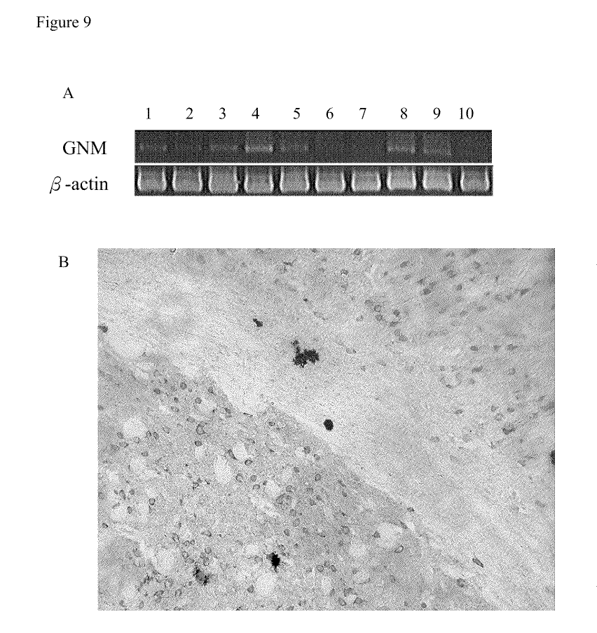

Isolated brain tissues of WT mice are fixed in 10% neutral-buffered formalin. After infiltrating with 30% sucrose solution in PBS, cut the tissue using frozen sections and paraffin sections (method). For antigen retrieval, tissue sections on slides were immersed in borate buffer solution (pH 8) jar and placed in pressure oven for about 20 min until the cooker reached its maximum pressure. It was then heated for another 5 min at maximum pressure. Thereafter, the pressure was reduced and cooled in a bath of tap water. Then the sections incubate in blocking solution at room temperature for 6 hours. And they were incubated overnight at 4° C. with the following rabbit anti-GNMT sera at 1/100. After washing in PBS, these slides were incubated with biotinylated antibody and peroxidase-labeled streptavidin (DAKO, Carpinteria, Calif.) for 10 min at room temperature. These slides were further incubated with 3,3′-diaminobenzidine tetrahydrochloride solution for color reaction. (FIG. 9)

### Example 11

### Statistical Analysis

All data were pooled according to genotype, and a mean value was determined for each group. Results were presented as means±SEM and were analyzed by Student's t-test with p<0.05 used as significance criteria.

One skilled in the art readily appreciates that the present invention is well adapted to carry out the objects and obtain the ends and advantages mentioned, as well as those inherent therein. The cell lines, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An animal model for studying depression, schizophrenia or Alzheimer's disease, wherein the animal model is a rodent whose genome is disrupted by recombination at Glycine N-methyltransferase (GNMT) gene locus, and exhibiting a pathological condition of depression, schizophrenia or Alzheimer's disease.

2. (canceled)

3. The animal model of claim 1, wherein the rodent is mouse.

4. A method of generating the animal model of claim 1 with disruption of GNMT gene by recombination at GNMT gene locus, comprising introducing a genetic construct comprising a disruption such that function GNMT is not expressed from said gene into embryonic stein cells; screening for cells comprising the disrupted GNMT gene, in which recombination has occurred between the genetic construct and the endogenous gene; injecting the embryonic stem cell into a rodent blastocyst; transferring the blastocyst to pseudopregnant mouse; and allowing the transferred blastocyst to develop into a mouse chimeric for the disruption.

5-7. (canceled)

8. The method of claim 4, wherein the animal is mouse.

9. A method for screening a drug candidate for treating depression, schizophrenia or Alzheimer's disease in a subject, comprising:
(a) administering a potential drug candidate to the animal model of claim 1,
(b) measuring the response of said animal to said drug candidate,
(c) comparing the response of said animal with that of an animal having a wild type GNMT gene, and
(d) selecting the drug candidate based on the difference in response observed between said animal and said animal having a wild type GNMT gene.

10. The method of claim 9, wherein the response is acoustic startle reflex, tail suspension test, or forced swim test.

11. The animal model of claim 1, wherein the pathological condition is characterized by deficits in prepulse inhibition of acoustic startle reflex, decreased immobility of tail suspension test and forced swim test, or elevating expression of Alzheimer's disease-associated genes.

12. The animal model of claim 12, wherein the Alzheimer's disease-associated genes are BACE 1, BACE 2, APH-1, GSK-3, MAPT, and IDE.

13. The method of claim 4, wherein the genetic construct comprising a positive selection marker flanked by segments showing sufficient sequence relatedness to the GNMT gene to undergo homologous recombination with it.

14. The method of claim 4, wherein the screened embryonic stem cell is homozygous for the deletion of the GNMT gene.


SEQUENCE LISTING (26 sequences)

SEQ ID NO: 1 - DNA, 29 bp, Artificial Sequence
  GNMT-F
gcggcggccgcatgctggtggaagagggc

SEQ ID NO: 2 - DNA, 20 bp, Artificial Sequence
  GNMT-R
ttgcagtctggcaagtgagc

SEQ ID NO: 3 - DNA, 20 bp, Artificial Sequence
  neomycin-F
gttccttgcgcagctgtgct

SEQ ID NO: 4 - DNA, 20 bp, Artificial Sequence
  neomycin-R
cggccacagtcgatgaatcc

SEQ ID NO: 5 - DNA, 29 bp, Artificial Sequence
  m-GNMT-F
gcggcggccgcatgctggtggaagagggc

SEQ ID NO: 6 - DNA, 20 bp, Artificial Sequence
  m-GNMT-R
ttgcagtctggcaagtgagc

SEQ ID NO: 7 - DNA, 17 bp, Artificial Sequence
  beta-actin-F
gggcgccccaggcacca

SEQ ID NO: 8 - DNA, 23 bp, Artificial Sequence
  beta-actin-R
ctccttaatgtcacgccgatttc

SEQ ID NO: 9 - DNA, 20 bp, Artificial Sequence
  APP-F
gccagccaataccgaaaatg

SEQ ID NO: 10 - DNA, 22 bp, Artificial Sequence
  APP-R
gatgtttgtcagcccagaacct

SEQ ID NO: 11 - DNA, 21 bp, Artificial Sequence
  BACE1-F
acgactctttggagcccttct

SEQ ID NO: 12 - DNA, 22 bp, Artificial Sequence
  BACE1-R
agagctgcagggaaaagatgtt

SEQ ID NO: 13 - DNA, 21 bp, Artificial Sequence
  BACE2-F
cacggaagacatagccagcaa

SEQ ID NO: 14 - DNA, 21 bp, Artificial Sequence
  BACE2-R
tcagggcataggacacaatcc

SEQ ID NO: 15 - DNA, 20 bp, Artificial Sequence
  IDE-F
cgtccaatctgatggcgatt

SEQ ID NO: 16 - DNA, 23 bp, Artificial Sequence
  IDE-R
agaacagcttcaccaccaggtta

SEQ ID NO: 17 - DNA, 32 bp, Artificial Sequence
  SNCA-F
aaacacctaagtgactaccacttatttctaaa

SEQ ID NO: 18 - DNA, 24 bp, Artificial Sequence
  SNCA-R
tcttggagcaaatcacaacttctt

SEQ ID NO: 19 - DNA, 25 bp, Artificial Sequence
  MAPT-F
agcaatgagagatttgagacttggt

SEQ ID NO: 20 - DNA, 19 bp, Artificial Sequence
  MAPT-R
ccttcgctgtcgctgtttc

SEQ ID NO: 21 - DNA, 19 bp, Artificial Sequence
  APH1 alpha-F
atgcacggctccagtatgg

SEQ ID NO: 22 - DNA, 23 bp, Artificial Sequence
  APH1 alpha-R
gcaaaacggaacacttcctgtag

SEQ ID NO: 23 - DNA, 20 bp, Artificial Sequence
  GSK3 beta-F
cgggacccaaatgtcaaact

SEQ ID NO: 24 - DNA, 20 bp, Artificial Sequence
  GSK3 beta-R
tccgagcatgtggagggata

SEQ ID NO: 25 - DNA, 20 bp, Artificial Sequence
  GAPDH-F
tggtatcgtggaaggactca

SEQ ID NO: 26 - DNA, 20 bp, Artificial Sequence
  GAPDH-R
agtgggtgtcgctgttgaag